SEQUENCE LISTING (9 sequences)

SEQ ID NO: 1 - protein, 1037 aa, Artificial Sequence
  AlphaIIb monomeric subunit
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERGGGSGGGSGGGSDYKDDDDKGDYKDDDDKIDYKDDDDKHHHHHH

SEQ ID NO: 2 - protein, 993 aa, Artificial Sequence
  Alpha IIb moiety
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEER

SEQ ID NO: 3 - protein, 12 aa, Artificial Sequence
  Linker for AlphaIIb
GGGSGGGSGGGS

SEQ ID NO: 4 - protein, 26 aa, Artificial Sequence
  Acidic tail for AlphaIIb
DYKDDDDKGDYKDDDDKIDYKDDDDK

SEQ ID NO: 5 - protein, 6 aa, Artificial Sequence
  Histidine tag for AlphaIIb
HHHHHH

SEQ ID NO: 6 - protein, 744 aa, Artificial Sequence
  Beta3 monomeric subunit
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDHPGGGSGGGSGGGSWKKYKKSDPGWKKYKKSDGGWKKYKKSDPGWKKYKWSD

SEQ ID NO: 7 - protein, 692 aa, Artificial Sequence
  Beta3 moiety
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPD

SEQ ID NO: 8 - protein, 14 aa, Artificial Sequence
  Linker for Beta3
HPGGGSGGGSGGGS

SEQ ID NO: 9 - protein, 79 aa, Artificial Sequence
  Basic tail for Beta3
RFQYYEDSSGKSILYVVEEPECPKGPDHPGGGSGGGSGGGSWKKYKKSDPGWKKYKKSDGGWKKYKKSDPGWKKYKWSD

(19) United States
(12) Patent Application Publication
(10) Pub. No.: US 2023/0331811 A1
(43) Pub. Date: Oct. 19, 2023

(54) IMMOBILIZED SELF-ASSEMBLED PROTEIN MULTIMERS

(71) Applicant: CCOA THERAPEUTICS INC., Toronto (CA)

(72) Inventors: Heyu NI, North York (CA); Miguel NEVES, São Victor, Braga (PT)

(21) Appl. No.: 17/997,090

(22) PCT Filed: Apr. 27, 2021
(86) PCT No.: PCT/CA2021/050573

§ 371 Date: Oct. 25, 2022

Related U.S. Application Data
(60) Provisional application No. 63/015,781, filed on Apr. 27, 2020.

(51) Int. Cl. C07K 14/705; C07K 17/14

(52) U.S. Cl. CPC C07K 14/70546; C07K 17/14; C07K 2319/03

(57) ABSTRACT

Surfaces and processes are provided for immobilization of multimer polypeptides that preserves the functionality and active conformation of the native multimeric polypeptides. The multimer polypeptide is a self-assembled multimer and comprises both a first and a second chimeric polypeptide.

### TECHNOLOGICAL FIELD

The present disclosure relates to surfaces having a polypeptide multimer, such as a polypeptide heterodimer, immobilized thereon and maintaining the biological activity of the heterodimer.

### BACKGROUND

Immobilized proteins are important tools in proteomics and diagnosis, allowing one to obtaining information about protein functions and interactions. Immobilized proteins and enzymes are very important for commercial uses as they possess many benefits to the expenses and processes of the associated reaction, including convenience of use, reusability, and greater stability compares to soluble forms of the proteins and enzymes. Ideally, proteins need to be immobilized onto surfaces while maintaining their biological activity, optionally with high density in order to allow the usage of small amount of sample solution. Nonspecific protein adsorption also need to be avoided or at least minimized in order to improve detection performances.

Although various approaches to immobilizing proteins are available, considerable development is still required, especially for full retention of protein conformation, of heterodimers for example, and activity which remains a challenge.

### SUMMARY

The present disclosure provides biologically active forms of multimers (such as heterodimers) on a surface using charged tails. The charged tails immobilizes the multimers onto the surface, while still allowing the monomeric protein units of the multimers to interact with each other. The charged tails also interact with one another.

In accordance with an aspect, there is provided a surface having a first and a second hydroxyl group and at least one self-assembled multimer immobilized thereon, wherein: the at least one self-assembled multimer comprises at least one a first chimeric polypeptide associated with a second chimeric polypeptide. The first chimeric polypeptide is of formula (Ia) or (Ib), the second chimeric polypeptide is of formula (IIa) or (IIb):

NH₂-FPM-FAAL-AAT-COOH (Ia)

NH₂-AAT-FAAL-FPM-COOH (Ib)

wherein FPM is a first polypeptide moiety; FAAL is an optional first amino acid linker; AAT is an acidic amino acid tail having at least three acidic amino acid residues each having an R-group comprising a carboxyl group, and wherein the AAT has a pI between about 3 and about 5; and — is an amine bond. The second chimeric polypeptide is of formula (IIa) or (IIb):

NH₂-SPM-SAAL-BAT-COOH (IIa)

NH₂-BAT-SAAL-SPM-COOH (IIb)

wherein SPM is a second polypeptide moiety; SAAL is an optional second amino acid linker; BAT is a basic amino acid tail having at least one acid amino acid residue having an R-group comprising a carboxyl group, and wherein the BAT has a pI between about 9 and 11; and — is an amine bond. The carboxyl group of the first chimeric polypeptide is covalently associated to a first silane linker (FSL) moiety, wherein the FSL is covalently associated with a first hydroxyl group of the surface. The carboxyl group of the second chimeric polypeptide is covalently associated to a second silane linker (SSL) moiety, wherein the SSL is covalently associated with a second hydroxyl group of the surface. The AAT is non-covalently associated with the BAT. The first chimeric polypeptide is non-covalently associated with the second polypeptide moiety. In some embodiments, the FPM and the SPM are the same and the multimer is a homodimer, a homotrimer or a homomultimer comprising more than three identical polypeptide moieties. In additional embodiments, the FPM and the SPM are different and the multimer is an heterodimer, a heterotrimer or a heteromultimer comprising additional polypeptide moieties. In an embodiment, the AAT is at least three and up to 50 amino acid residues in length. In a further embodiment, wherein the AAT has a pI is about 4. In yet another embodiment, wherein the AAT has a pI of about 3.91. In some embodiments, the AAT has an amino acid sequence of SEQ ID NO: 4 or functional variants or fragments thereof. In an embodiment, the BAT is at least one and up to 50 amino acid residues in length. In a further embodiment, the BAT has a pI is about. In yet a further embodiment, the BAT has a pI of about 10.1. In still another embodiment, the BAT has an amino acid sequence of SEQ ID NO: 9 or functional variants or fragments thereof. In yet another embodiment, the first chimeric polypeptide has the FAAL and/or the second chimeric polypeptide has the SAAL. In an embodiment, the at least one self-assembled multimer comprising an ectodomain of a surface protein. In still another embodiment, one or more of the at least one self-assembled multimer is an activated surface protein. In still another embodiment, the at least one self-assembled multimer is an integrin dimer. In another embodiment, the first chimeric polypeptide comprises a αIIb polypeptide, and the second chimeric polypeptide comprises a β3 polypeptide. In such embodiment, the FPM has an amino acid sequence of SEQ ID NO: 2 or functional variants or fragments thereof and/or the SPM has an amino acid sequence of SEQ ID NO: 7 or functional variants or fragments thereof. In an embodiment, the surface is a spherical surface, such as, for example, a microsphere (e.g., a microsphere silica bead). In still another embodiment, the surface comprises a planar surface. In some embodiments, the FSL and/or SSL comprise one or more amine or thiol groups that are covalently associated with the carboxyl groups of the AAT or the BAT. In still further embodiments, the FSL and/or SSL moieties comprise (3-trimethoxysilylpropyl) diethylenetriamine (DETA). In specific embodiments, the surface comprises the first chimeric polypeptide of formula (Ia) and the second chimeric polypeptide of formula (IIa); the first chimeric polypeptide of formula (Ib) and the second chimeric polypeptide of formula (IIa); the first chimeric polypeptide of formula (Ia) and the second chimeric polypeptide of formula (IIb); or the first chimeric polypeptide of formula (Ib) and the second chimeric polypeptide of formula (IIb).

In accordance with another aspect, there is provided a process of immobilizing at least one self-assembled multimer to a surface having a first and a second hydroxyl groups covalently associated with a first and a second silane linker moiety. The at least one self-assembled multimer comprises a first and a second chimeric polypeptide. The process comprises obtaining the first chimeric polypeptide as described herein; obtaining the second chimeric polypeptide as described herein; and adding the first and the second chimeric polypeptide to the surface in a solvent under suitable conditions for the first and second chimeric polypeptides to covalently bond to the surface via the silane linker moieties. The AAT of the first chimeric polypeptide is non-covalently associated with the BAT of the second chimeric polypeptide and the first polypeptide moiety is non-covalently associated with the second polypeptide moiety. The process of claim 24, wherein the first and second silane linker moieties comprise one or more amine or thiol groups that are covalently associated with the carboxyl groups of the AAT or BAT. In an embodiment, the first and/or second silane linker moieties comprise (3-trimethoxysilylpropyl) diethylenetriamine (DETA). In another embodiment, the process further comprises coating the surface with the silane linker moieties by reacting with the hydroxyl groups. In still another embodiment, the process further comprises obtaining the first and the second chimeric polypeptide from recombinant expression in a recombinant host cell. In still another embodiment, the process further comprises activating the at least one self-assembled multimer. In such embodiment, the process comprises incubating the surface having the first and second chimeric polypeptides bonded thereon in an activation buffer comprising cations. In some embodiments, the activation buffer comprises divalent cations. In a further embodiment, the at least one self-assembled multimer comprises an ectodomain of a surface protein. In an embodiment, the at least one self-assembled multimer is an integrin dimer. In still another embodiment, the surface is a microsphere or is flat.

In accordance with another aspect, there is provided a kit comprising (i) a first and (ii) a second chimeric polypeptide as described herein, wherein the first and the second chimeric polypeptide are capable of forming a multimer and optionally (iii) a surface for covalently associating the first and the second chimeric polypeptide, wherein the surface has hydroxyl groups covalently associated with a first and a second silane linker moiety. In an embodiment, the first chimeric polypeptide has a first polypeptide moiety (FPM), an optional first amino acid linker (FAAL), and a first amino acid tail (AAT) having at least three acid amino acid residue having an R-group comprising a carboxyl group, and wherein the AAT has a pI between about 3 and 5 and the second chimeric polypeptide has a second polypeptide moiety (SPM), an optional second amino acid linker (SAAL), and a second amino acid tail (BAT) having at least one acid amino acid residue having an R-group comprising a carboxyl group, and wherein the BAT has a pI between about 9 and 11. In an embodiment, the FPM is a αIIb polypeptide and the SPM is a β3 polypeptide. In such embodiment, the first chimeric polypeptide can have an amino acid sequence of SEQ ID NO: 1 or functional variants or fragments thereof and/or the second chimeric polypeptide can have an amino acid sequence of SEQ ID NO: 6 or functional variants or fragments thereof. In some embodiments, the surface is a microsphere, such as, for example, a microsphere silica bead (which can be coated with the silane linker moiety). In still another embodiment, the surface is flat (which can be coated with the silane linker moiety). In an embodiment, the silane linker moieties comprise one or more amine or thiol groups that are covalently associated with the carboxyl groups of the AAT or BAT. In still another embodiment, the silane linker moiety is (3-trimethoxysilylpropyl) diethylenetriamine (DETA).

### DESCRIPTION OF THE FIGURES

Having thus generally described the nature of the invention, reference will now be made to the accompanying drawings, showing by way of illustration, a preferred embodiment thereof, and in which:

FIGS. 1A to 1C (FIG. 1A) Generalized representation of unimolecular self-assembled monolayer (SAM) surface assembly. Linkers consisting of a backbone, flanked by a tail and a head group, assemble with the tails attached to the silica and the heads available for covalent probe attachment. (FIG. 1B) The different conformation states of an integrin are presented. In the inactive (“Bent”) and intermediate (“Extended”) conformations, integrins have no/low affinity towards their target. In the high-affinity ligand binding (“Open”) conformation, integrins have affinity towards their target. For each pair of heterodimer shown, left heterodimer is the α-subunit and the right heterodimer is the β-subunit. (FIG. 1C) Illustration of the formation of a (3-trimethoxysilylpropyl) diethylenetriamine (DETA) SAM onto a cleaned silica substrate (step 1), coupling of recombinant human ectodomain αIIbβ3 onto DETA-SAM (step 2) which involves site-specific coupling of recombinant human ectodomain αIIbβ3 having acidic and basic tails onto DETA SAMs (step 2a), and hydrolysis of any unreacted NHS groups to stop the surface coupling reaction by immersing the substrate in borate buffer pH 8.5 (step 2b). Activation of immobilized recombinant human ectodomain αIIbβ3 into high-affinity ligand binding conformation (step 3). For each pair of heterodimer shown, left heterodimer is αIIb and the right heterodimer is β3.

FIGS. 2A to 2I show the characterization of αIIbβ3-coupled silica particles by comparing bare, DETA coated, inactive αIIbβ3 coupled and activated αIIbβ3 coupled beads with fluorescently coupled antibodies and ligands. (FIG. 2A) flow cytometry histograms showing PSI-E1 binding (conformation independent αIIbβ3 mAb), (FIG. 2B) corresponding mean fluorescence intensities (MFI) from FIG. 2A plotted in bar graphs, (FIG. 2C) flow cytometry histograms showing fibrinogen (endogenous ligand of αIIbβ3) binding, (FIG. 2D) corresponding mean fluorescence intensities (MFI) from FIG. 2C plotted in bar graphs, (FIG. 2E) fibrinogen dose-response curves, (FIG. 2F) flow cytometry dot plots and corresponding MFI bar graphs of DETA and activated αIIbβ3 particles binding fibrinogen before and after treatment with 1 mM EDTA, (FIG. 2G) flow cytometry dot plots of (left to right) DETA, Pre-activation and activated αIIbβ3 particles analyzed for PAC-1 binding, (FIG. 2H) corresponding mean fluorescence intensities (MFI) from FIG. 2G plotted in bar graphs, (FIG. 2I) bar graph of percentage of DETA, Pre-activation and, activated αIIbβ3 particles in the PAC-1 positive binding population of FIG. 2G. In each flow cytometry histogram, the fluorescent intensity is indicated on the X-axis, and the number of events is indicated on the Y-axis. In each bar graph, the particle type is indicated on the X-axis, and the mean fluorescent intensity (MFI) is indicated on the Y-axis. In each dose-response curve, concentration is indicated on the X-axis, and mean fluorescent intensity (MFI) is indicated on the Y-axis. In each dot plots, fluorescent intensity is indicated on the X-axis, and the side scatter is indicated on the Y-axis. In the bar graph of FIG. 2I, the particle type is indicated on the X-axis, and percentage population is indicated on the Y-axis.

FIGS. 3A and 3B show αIIbβ3 is covalently bound to the surface of the beads and covalent binding increases the anti-fouling and fibrinogen binding properties of the αIIbβ3-coupled beads. (FIG. 3A) DETA coated, αIIbβ3 coupled beads with FITC-coupled PSI-E1 (conformation independent αIIbβ3 mAb) in the absence (left panel) and presence (right panel) of SDS. (FIG. 3B) DETA coated, αIIbβ3 coupled beads with fibrinogen (endogenous ligand of αIIbβ3). Results are provided as the mean fluorescence intensity on the Y-axis, and adsorbed or covalent αIIbβ3 (as well as active or inactive conformation) on the X-axis.

FIGS. 4A and 4B show co-aggregation of αIIbβ3-coupled beads with (FIG. 4A) wild type and (FIG. 4B) Fibrinogen/von Willebrand factor double deficient (Fg/VWF^{-/-}) gel-filtered platelets. White arrows indicates platelets and black arrows indicates αIIbβ3 coated beads. Scale bar 20 μm.

FIGS. 5A and 5B compares (FIG. 5A) quantitative flow cytometry fibrinogen assay versus (FIG. 5B) fibrinogen ELISA. n≥3. In FIG. 5A, X-axis is fibrinogen concentration in μM, Y-axis is mean fluorescence intensity. In FIG. 5B, X-axis is fibrinogen concentration in μM, Y-axis is absorbance at 492 nm.

FIG. 6 shows the optimization of the loading of recombinant human αIIbβ3 ectodomain onto the DETA coated silica surface by evaluating the amount of integrin loaded on the surface against the binding activity against its cognate ligand, fibrinogen. Results are shown as flow cytometry signal associate with fibrinogen binding (left axis, grey line and ▪) or with PSI-E1 binding (right axis, black line and ●) in function of the concentration of αIIbβ3 ectodomain immobilized onto the DETA coated silica surface.

FIGS. 7A to 7C. (FIG. 7A) Platelet aggregates form under the conditions used for the platelet-bead co-aggregation experiments. Left panel shows the bright field color. Right panel shows the bright field. (FIG. 7B) DETA beads in the presence of fibrinogen, and αIIbβ3 coated beads without fibrinogen do not aggregate. While αIIbβ3 coated beads in the presence of fibrinogen aggregate. Left set of panels shows the bright field color. Right set of panels shows bright field with fluorescence overlay to clearly show aggregates. (FIG. 7C) Representative micrographs of the aggregation of αIIbβ3 coated beads in the presence of fibrinogen. Scale bar=10 μm.

### DETAILED DESCRIPTION

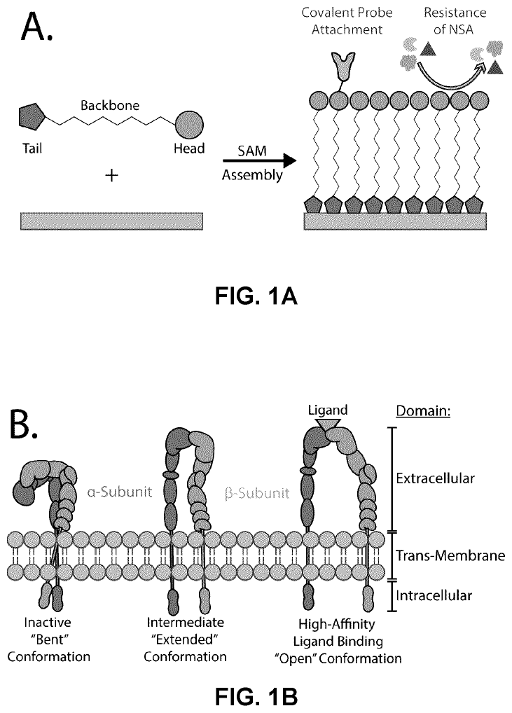

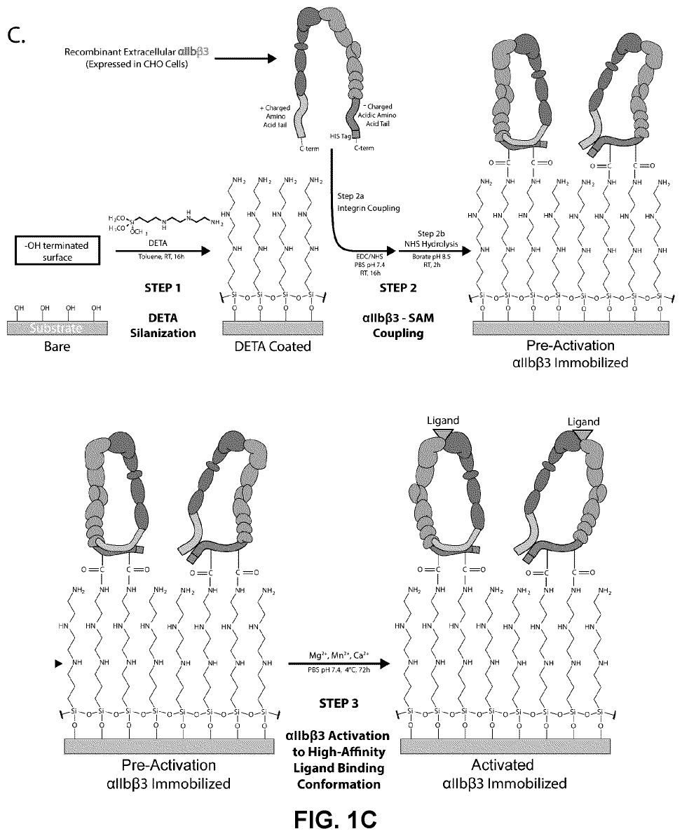

The present disclosure relates to multimeric polypeptides, such as homo- or heteromultimers, such as homo- or heterodimers (which can include, in some embodiments, one or more ectodomains), immobilized on surfaces that maintain their function and active conformation. The polypeptides are provided in the form of chimeric polypeptides having an amino acid tail for covalently associating with the surface, thereby immobilizing the chimeric polypeptides to the surface so as to form the multimer. FIG. 1A provides a representation of unimolecular self-assembly entities which can be combined to form the multimeric polypeptides.

As known in the art of dimeric polypeptides, like integrins, multimeric polypeptides can be provided in an inactive or “bent” conformation, an intermediate or “extended” conformation and, upon the binding of the cognate ligand, a high affinity or “open” conformation (FIG. 1B). The multimeric polypeptides of the present disclosure seek to be provided in an activable or an active confirmation.

In some embodiments, the amino acid tail has at least one acid amino acid residue having an R-group comprising a carboxyl group. As used herein, an “acid amino acid residue having an R-group comprising a carboxyl group” refers to natural or unnatural amino acids having a side chain R-group that has one or more terminal or non-terminal carboxyl group (—C(═O)O—), where the carboxyl group is capable of binding with an silane linker moiety. Examples of such natural amino acids ((L)-configuration) having a carboxyl R-group are aspartic acid and glutamic acid. Unnatural amino acids having a side chain R-group include, for example, amino acids with dextrorotary (D)-configuration or amino acids with synthetic or variant R-groups (termed non-natural amino acids) that have been modified to add a terminal or non-terminal carboxyl group. The person skilled in the art will recognize that the amino acid residues present on the tail of each of the chimeric polypeptide is not limited to a particular naturally-occurring or synthetic amino acid residues.

The amino acid tail is attached to the polypeptide (either directly or indirectly using a linker) in such a way that the polypeptide maintains its conformation, functionality or biological activity. In an embodiment, the amino acid tail is attached (either directly or indirectly using a linker) to one end (carboxyl- or amino-end) of the polypeptide. In some embodiments, the amino acid tail is attached to an end of the polypeptide that is opposite to the functional end of the polypeptide to avoid loss of conformation, functionality or biological activity of the polypeptide. For example, if the polypeptide bears its biological activity at the carboxyl-end, the amino acid tail is going to be attached to the amino-end of the polypeptide. In another example, if the polypeptide gears its biological activity at the amino-end, the amino acid tail is going to be attached to the carboxyl-end of the polypeptide. In some embodiments, the amino acid tail is attached to the carboxyl end of the polypeptide. In other embodiments, the amino acid tail is attached to the amino end of the polypeptide.

In order to immobilize the polypeptide on the surface, one of the carboxyl group of the polypeptide (which can be associated with the amino acid tail) is covalently associated with a silane linker. In some embodiments, the carboxyl group of the R-group of one of the amino acid residue of an amino acid tail is for covalent association (a chemical bond) with a silane linker moiety which is immobilized on the surface. In some embodiments, the silane linker moiety has one or more terminal or non-terminal the amine (—NH₂—) or thiol (—S—) groups for covalent association or chemical bonding with the carboxyl group of the polypeptide and, in some embodiments, of the acidic amino acid residue(s) of the amino acid tail associated to the polypeptide.

The polypeptides of the present disclosure can be presented as chimeric polypeptides. In the context of the present disclosure, the chimeric polypeptides can have formula (IIIa) or (IIIb):

NH₂-PM-AAL-AT-COOH (IIIa)

NH₂-AT-AAL-PM-COOH (IIIb)

wherein PM is a polypeptide moiety (which can include an ectodomain), AAL is an optional amino acid linker, and AT is an amino acid tail.

In some embodiments, the chimeric polypeptides can have formula (IVa) or (IVb):

NH₂-PM-AT-COOH (IVa)

NH₂-AT-PM-COOH (IVb)

wherein PM is a polypeptide moiety (which can include an ectodomain) and AT is an amino acid tail.

When the amino acid linker (AAL) is absent, the amino acid tail is directly associated with the polypeptide moiety. In the chimeric polypeptide of formula (IVa), this means that the carboxyl terminus of the polypeptide moiety is directly associated (with an amide linkage) to the amino terminus of the amino acid tail. In the chimeric polypeptide of formula (IVb), this means that the carboxyl terminus of the amino acid tail is directly associated (with an amide linkage) to the amino terminus of the polypeptide moiety.

In some embodiments, the presence of an amino acid linker (AAL) is desirable either to provide, for example, some flexibility between the polypeptide moiety and the amino acid tail or to facilitate the construction of the chimeric polypeptide (which can, in some embodiments, be encoded by a nucleic acid molecule). As used in the present disclosure, the “amino acid linker” or “AAL” refer to a stretch of one or more amino acids separating the polypeptide moiety (PM) and the amino acid tail (AT) (e.g., indirectly linking the polypeptide moiety to the amino acid tail). It is preferred that the amino acid linker be neutral, e.g., does not interfere with the biological activity of the polypeptide moiety nor with the biological or chemical activity or interactions of the amino acid tail.

In instances in which the amino acid linker (AAL) is present in the chimeras of formula (IIIa or IVa), its amino end is associated (with an amide linkage) to the carboxyl end of the polypeptide moiety and its carboxyl end is associated (with an amide linkage) to the amino end of the amino acid tail. In instances in which the amino acid linker (AAL) is present in the chimeras of formula (IIIb of IVb), its amino end is associated (with an amide linkage) to the carboxyl end of the amino acid tail and its carboxyl end is associated (with an amide linkage) to the amino end of the polypeptide moiety.

Various amino acid linkers exist and include, without limitations, (G)ₙ, (GS)ₙ; (GGS)ₙ; (GGGS)ₙ; (GGGGS)ₙ; (GGSG)ₙ; (GSAT)ₙ, wherein n = is an integer between 1 to 8 (or more). In an embodiment, the amino acid linker is (GGGS)ₙ (also referred to as G3S) and in still further embodiments, the amino acid linker L comprises more than one G3S motifs. For example, the amino acid linker can be (G₃S)₃ and have the amino acid sequence of SEQ ID NO 3.

In some embodiments, the amino acid tail (AT) is at least one amino acid and up to 50 amino acid residues in length. In some embodiments, the amino acid tail (AT) is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues long. In some embodiments, the amino acid tail is no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues long.

### Chimeric Multimer Polypeptides

In the context of the present disclosure, chimeric multimer polypeptides are provided to immobilize multimers on surfaces while maintaining their function and, in some embodiments, active conformation with respect to each other. In some embodiments, a surface is provided having hydroxyl groups and at least one self-assembled multimer immobilized thereon. The multimer comprises at least one first chimeric polypeptide associated with a second chimeric polypeptide. The multimer can be a homo-multimer or a hetero-multimer.

In some embodiments, the first chimeric polypeptide is of formula (Ia) or (Ib):

NH₂-FPM-FAAL-AAT-COOH (Ia)

NH₂-AAT-FAAL-FPM-COOH (Ib)

wherein FPM is a first polypeptide moiety; FAAL is an optional first amino acid linker; AAT is an acidic amino acid tail having at least three acidic amino acid residues having an R-group comprising a carboxyl group. The AAT has a pI between about 3 and 5; and — is an amine bond.

In some embodiments, the first chimeric polypeptide is of formula (Va) or (Vb):

NH₂-FPM-AAT-COOH (Va)

NH₂-AAT-FPM-COOH (Vb)

wherein FPM is a first polypeptide moiety; AAT is an acidic amino acid tail having at least three acidic amino acid residues having an R-group comprising a carboxyl group. The AAT has a pI between about 3 and 5. The — is an amine bond.

In some embodiments, the FPM includes an ectodomain (and in some additional embodiments, a complete ectodomain) of a surface polypeptide. As it is known in the art, an ectodomain is the domain of a membrane protein that extends in the extracellular space. In some embodiments, the ectodomain is involved in binding a ligand and can lead to signal transduction.

As used herein, an “acidic amino acid tail” refers to an amino acid tail having one or more acidic amino acid residues, such that the pI of the acidic amino acid tail is less than 7. Examples of acidic amino acid residues include: aspartic acid and glutamic acid. In some embodiments, the pI of the acidic amino acid tail is less between about 3 and 5. In one embodiment, the pI of the acidic amino acid tail is about 4, more preferably 3.91. The acidic amino acid tail has a charge which will allow it to interact non-covalently with the basic amino acid tail (described below) and place the FPM is an active conformation.

In some embodiments, the acidic amino acid tail (AAT) is at least three amino acid and up to 50 amino acid residues in length. In some embodiments, the acidic amino acid tail (AAT) is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues long. In some embodiments, the acidic amino acid tail is no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4 or 3 amino acid residues long. In some embodiments, the AAT has an amino acid sequence of SEQ ID NO: 4 or variants or fragments thereof.

In instances in which the first amino acid linker (FAAL) is present in the chimeras of formula (Ia), its amino end is associated (with an amide linkage) to the carboxyl end of the first polypeptide moiety and its carboxyl end is associated (with an amide linkage) to the amino end of the acidic amino acid tail. In instances in which the first amino acid linker (FAAL) is present in the chimeras of formula (IIb), its amino end is associated (with an amide linkage) to the carboxyl end of the acidic amino acid tail and its carboxyl end is associated (with an amide linkage) to the amino end of the first polypeptide moiety.

When the first amino acid linker (FAAL) is absent, the acidic amino acid tail is directly associated with the first polypeptide moiety. In the chimeric polypeptide of formula (Va), this means that the carboxyl terminus of the first polypeptide moiety is directly associated (with an amide linkage) to the amino terminus of the acidic amino acid tail. In the chimeric polypeptide of formula (Vb), this means that the carboxyl terminus of the acidic amino acid tail is directly associated (with an amide linkage) to the amino terminus of the first polypeptide moiety.

In some embodiments, the second chimeric polypeptide is of formula (IIa) or (IIb):

NH₂—SPM-SAAL-BAT-COOH (IIa)

NH₂-BAT-SAAL-SPM-COOH (IIa)

wherein SPM is a second polypeptide moiety; SAAL is an optional second amino acid linker; BAT is a basic amino acid tail having at least one acid amino acid residue having an R-group comprising a carboxyl group. The BAT has a pI between about 9 and 11. The — is an amine bond. It is understood that the SPM can differ from the FPM and that the SPM can be capable of forming an heterodimer or an heterotrimer with the FPM. In some embodiments, the FPM and the SPM can be identical or substantially similar so as to form a homodimer or a homotrimer for example.

In some embodiments, the second chimeric polypeptide is of formula (VIa) or (VIb):

NH₂-SPM-BAT-COOH (VIa)

NH₂-BAT-SPM-COOH (VIa)

wherein SPM is a second polypeptide moiety; BAT is a basic amino acid tail having at least one acid amino acid residue having an R-group comprising a carboxyl group. The BAT has an isoelectric point (pI) between about 9 and 11. The — is an amine bond.

In some embodiments, the SPM includes an ectodomain (and in some additional embodiments, a complete ectodomain) of a surface polypeptide. As it is known in the art, an ectodomain is the domain of a membrane protein that extends in the extracellular space. In some embodiments, the ectodomain is involved in binding a ligand and can lead to signal transduction.

As used herein, a “basic amino acid tail” refers to an amino acid tail having one or more basic amino acid residues, such that the pI of the basic amino acid tail is greater than 7. Examples of basic amino acid residues include: arginine, histidine, and lysine. In some embodiments, the pI of the basic amino acid tail is less between about 9 and 11. In one embodiment, the pI of the acidic amino acid tail is about 10, more preferably 10.05. The basic amino acid tail has a charge which will allow it to interact non-covalently with the acidic amino acid tail and place the SPM is an active conformation.

In some embodiments, the basic acid tail (BAT) is at least one amino acid and up to 50 amino acid residues in length. In some embodiments, the basic amino acid tail (BAT) is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues long. In some embodiments, the basic amino acid tail is no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues long. In some embodiments, the BAT has an amino acid sequence of SEQ ID NO: 9 or variants or fragments thereof.

In instances in which the second amino acid linker (SAAL) is present in the chimeras of formula (IIa), its amino end is associated (with an amide linkage) to the carboxyl end of the second polypeptide moiety and its carboxyl end is associated (with an amide linkage) to the amino end of the basic amino acid tail. In instances in which the second amino acid linker (SAAL) is present in the chimeras of formula (IIb), its amino end is associated (with an amide linkage) to the carboxyl end of the basic amino acid tail and its carboxyl end is associated (with an amide linkage) to the amino end of the second polypeptide moiety.

When the second amino acid linker (SAAL) is absent, the basic amino acid tail is directly associated with the second polypeptide moiety. In the chimeric polypeptide of formula (VIa), this means that the carboxyl terminus of the second polypeptide moiety is directly associated (with an amide linkage) to the amino terminus of the basic amino acid tail. In the chimeric polypeptide of formula (VIb), this means that the carboxyl terminus of the basic amino acid tail is directly associated (with an amide linkage) to the amino terminus of the second polypeptide moiety.

To immobilize chimeric polypeptides to the surface, one or more of the carboxyl group of the acidic amino acid tail (AAT) of the first chimeric polypeptide is covalently associated to a first silane linker (FSL) moiety, which in turn is covalently associated with a first hydroxyl group of the surface. One or more of the carboxyl group of the basic amino acid tail (BAT) of the second chimeric polypeptide is covalently associated to a second silane linker (SSL) moiety, which in turn is covalently associated with a second hydroxyl group of the surface. When immobilized on the surface, the acidic amino acid tail (AAT) is non-covalently associated (such as charge-charge interaction) with the basic amino acid tail (BAT). Furthermore, the first polypeptide moiety is non-covalently associated with the second polypeptide moiety. In some embodiments, the first polypeptide moiety is non-covalently associated with the second polypeptide moiety in an active conformation.

In some embodiments, the first chimeric polypeptide has the FAAL, and/or the second chimeric polypeptide has the SAAL. In some embodiments, the first chimeric polypeptide has the FAAL, and the second chimeric polypeptide has the SAAL. In some embodiments, the first chimeric polypeptide has the FAAL, but the second chimeric polypeptide does not have the SAAL. In some embodiments, the first chimeric polypeptide does not have the FAAL, but the second chimeric polypeptide has the SAAL.

In some embodiments, the first chimeric polypeptide is of formula (Ia) and the second chimeric polypeptide is of formula (IIa). In some embodiments, the first chimeric polypeptide is of formula (Ib) and the second chimeric polypeptide is of formula (IIa). In some embodiments, the first chimeric polypeptide is of formula (Ia) and the second chimeric polypeptide is of formula (IIb). In some embodiments, the first chimeric polypeptide is of formula (Ib) and the second chimeric polypeptide is of formula (IIb).

In some embodiments, the first chimeric polypeptide is of formula (Va) and the second chimeric polypeptide is of formula (VIa). In some embodiments, the first chimeric polypeptide is of formula (Vb) and the second chimeric polypeptide is of formula (VIa). In some embodiments, the first chimeric polypeptide is of formula (Va) and the second chimeric polypeptide is of formula (VIb). In some embodiments, the first chimeric polypeptide is of formula (Vb) and the second chimeric polypeptide is of formula (VIb).

In some embodiments, the at least one self-assembled multimer is a surface protein (which can include one or more ectodomains for the FPM and/or the SPM). Preferably, the at least one self-assembled multimer is an activated surface protein. In some embodiments, at least one self-assembled multimer is a platelet surface protein. In one embodiment, the at least one self-assembled multimer is an integrin dimer. In one embodiment, the first chimeric polypeptide comprises a αIIb polypeptide, functional variants or fragments thereof; and the second chimeric polypeptide comprises a β3 polypeptide, functional variants or fragments thereof.

In one embodiment, the FPM has an amino acid sequence of SEQ ID NO: 2 or variants or fragments thereof. In such embodiment, the corresponding first chimeric polypeptide can have, for example, the amino acid sequence of SEQ ID NO: 1 or variants thereof or fragments thereof. In another embodiment, the FPM has an amino acid sequence of SEQ ID NO; 7 or variants thereof or fragments thereof.

In one embodiment, the SPM has an amino acid sequence of SEQ ID NO: 2 or variants or fragments thereof. In another embodiment, the SPM has an amino acid sequence of SEQ ID NO: 7 or variants thereof or fragments thereof. In such embodiment, the corresponding second chimeric polypeptide can have, for example, the amino acid sequence of SEQ ID NO: 6 or variants thereof or fragments thereof.

A variant comprises at least one amino acid difference when compared to the amino acid sequence of the polypeptide polypeptide and exhibits a biological activity substantially similar to the native polypeptide. The polypeptide “variants” have at least 50%, 55%, 60%, 65%, 70%, 75%, 80%, 85%, 90%, 95%, 96%, 97%, 98% or 99% identity to the polypeptide described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested.

Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The polypeptide “variants” have at least 50%, 55%, 60%, 65%, 70%, 75%, 80%, 85%, 90%, 95%, 96%, 97%, 98% or 99% of the biological activity of the polypeptide described herein. One of the biological activity of the αIIb polypeptide is its ability to non-covalently associated with the β3 polypeptide and bind to its ligand (such as fibrinogen). One of the biological activity of the β3 polypeptide is its ability to non-covalently associated with the αIIb polypeptide and bind to its ligand (such as the fibrinogen). The biological of “variants” of the αIIb or the β3 can be assessed by various means known in the art, including, but not limited to antibody-based techniques (flow cytometry, ELISA assay for example) as well as microscopy techniques.

The variant polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of the enzyme.

The polypeptide can be a fragment of polypeptide or fragment of a variant polypeptide. A polypeptide fragment comprises at least one less amino acid residue when compared to the amino acid sequence of the possesses and still possess a biological activity substantially similar to the native full-length polypeptide or functional variants thereof Polypeptide “fragments” have at least at least 100, 200, 300, 400, 500, 600, 700, 800, 900 or more consecutive amino acids of the polypeptide or the polypeptide variant. The polypeptide “fragments” have at least 50%, 55%, 60%, 65%, 70%, 75%, 80%, 85%, 90%, 95%, 96%, 97%, 98% or 99% identity to the polypeptide described herein. The polypeptide “fragments” have at least 50%, 55%, 60%, 65%, 70%, 75%, 80%, 85%, 90%, 95%, 96%, 97%, 98% or 99% of the biological activity of the polypeptides and the functional fragments described herein. In some embodiments, fragments of the polypeptides can be employed for producing the corresponding full-length enzyme by peptide synthesis. Therefore, the fragments can be employed as intermediates for producing the full-length polypeptides.

### Organosilane Surfaces

In the context of the present disclosure, the carboxyl groups of the chimeric polypeptides are covalently associated to silane linker moieties, which in turn are covalently associated with hydroxyl groups of the surface. In some embodiments, the surface is made of a material such as silica, glass, metal, or plastics. In some embodiments, the surface has terminal hydroxyl groups. In other embodiments, the surface is chemically treated to add terminal hydroxyl groups. The grafting density of hydroxyl groups on the surface can be adjusted, as known in the art, so as to favor or allow the non-covalent association of the acidic and basic amino acid tails as well as the non-covalent association of the first polypeptide moiety and the second polypeptide moiety.

In some embodiments, the surface is curved. In one embodiment, the surface is spherical. In one embodiment, the surface is a microsphere. In one embodiment, the microsphere is a silica bead. In one embodiment, the microsphere is a glass bead. In one embodiment, the microsphere is a metal bead. In one embodiment, the microsphere is a plastic bead.

In some embodiments, the surface has a planar surface. In some embodiment, the surface is flat. In one embodiment, the surface is a film. In other embodiments, the surface is a platform. In additional embodiments, the surface is a flat silica surface, a flat glass surface, a flat plastic surface or a flat metal surface.

In some embodiments, the hydroxyl groups of the surface is covalently associated with the silicone atom of a silane linker moiety. A silane is an inorganic compound having the chemical formula, SiH₄. As used herein a “silane linker” refers to a compound based on SiH₄, where one or more of the hydrogens is substituted with a group having one or more terminal and/or non-terminal the amine (—NH₂—) or thiol (—S—) groups. The terminal and/or non-terminal the amine (—NH₂—) or thiol (—S—) groups of a silane linker moiety is covalently associated with the carboxyl groups of the acidic or basic tails.

In some embodiments, the silane linker moiety is an amino silane. In one embodiment, the amino silane is an amino alkyl silane. In one embodiment, the silane linker moiety is 3-trimethoxysilylpropyl) diethylenetriamine (DETA). In some embodiments, the silane linker moiety is an thiol silane. In one embodiment, the amino silane is a thiol alkyl silane.

In embodiments where the first chimeric polypeptide comprises a αIIb polypeptide, variants or fragments thereof; and the second chimeric polypeptide comprises a β3 polypeptide, variants or fragments thereof, the surface is a probe surface such as a αIIbβ3 coupled bead, film, or platform. The αIIbβ3 coupled bead, film, or platform has application as a molecular probe to identify integrin binding partners, and active conformation of the αIIbβ3 heterodimer is maintained to allow for binding with platelets to form platelet aggregates.

### Processes for Immobilizing Chimeric Multimer Polypeptides

In the context of the present disclosure processes of immobilizing at least one self-assembled multimer to a surface is provided. The surface has hydroxyl groups covalently associated with a first and a second silane linker moiety, and the at least one self-assembled multimer is a first and a second chimeric polypeptide. In some embodiments, the process includes obtaining a first chimeric polypeptide as described herein, obtaining a second chimeric polypeptide as described herein, and adding the first and second chimeric polypeptide to the surface in a solvent under suitable conditions for the first and second chimeric polypeptides to covalently bond to the surface via a silane linker moiety, wherein the first polypeptide moiety is non-covalently associated with the second polypeptide moiety. In some embodiments, the first polypeptide moiety and the second polypeptide moiety form a multimer in an active conformation.

In some embodiments, the process involves coating the surface with a silane linker by reacting with the hydroxyl groups of the surface. In some embodiments, the surface having hydroxyl groups are coated with a silane linker having one or more terminal and/or non-terminal amine (—NH₂—) or thiol (—S—) groups. In one embodiment, the silane linker is DETA and the process involves coating the surface with a DETA linker. The person skilled in the art will recognize that the silane linker is not limited to a particular linker and that linkers other than DETA can be used.

As it is known in the art, the process can include pre-treating the surface to provide hydroxyl groups to allow the silane linker to associate with the surface. This can be done, for example, by pre-treating the surface with piranha (70% H₂SO₄+30% of 30% H₂O₂), 20-40% NaOH/KOH, or another strong acid or base treatment. The person skilled in the art will recognize that any pre-treatment exposing hydroxyl groups on the surface can be used in the context of the present disclosure.

In some embodiments, the process involves the recombinant expression of the first and/or the second chimeric polypeptide in a recombinant host cell to obtain the first and/or the second chimeric polypeptide. In such embodiment, the process can also include a step of purifying, at least partially, the first and/or second chimeric polypeptide from the recombinant host. In some embodiments, the process involves recombinant expression of a surface protein, which can include its ectodomain. In one embodiment, the process involves the recombinant expression of an integrin dimer.

In some embodiments, the process involves activating the multimer form between the first and second chimeric polypeptide. For example, the process can include incubating the surface having the first and second chimeric polypeptides bonded thereon in an activation buffer (which can, in some embodiments, comprise cations). In some embodiments, the cations are divalent cations. In one embodiment, the cations are magnesium cations (Mg²⁺). In one embodiment, the cations are calcium cations (Ca²⁺). In one embodiment, the cations are manganese cations (Mn²⁺).

The present invention will be more readily understood by referring to the following examples which are given to illustrate the invention rather than to limit its scope.

### Example I—Immobilization of αIIbβ3 on Silica Beads

Materials. Unless otherwise specified, all reagents were purchased from Sigma-Aldrich™ and used as received. Furthermore, all buffers and aqueous solutions were prepared using ultrapure distilled deionized water (ddH₂O) with a measured resistivity ≥18.0 MΩ·cm.

DETA Silanization of Ferromagnetic Silica Beads and Subsequent Immobilization of αIIbβ3. Unless otherwise specified rinsing of ferromagnetic silica beads consists of magnetically pelleting the beads, removing the supernatant and resuspending in new solution. (—OH). Activated ferromagnetic silica beads were purchased from Magna Medics™ and used as received. The beads were first rinsed (3×) in spectral grade methanol, sonicated for 5 min then rinsed one last time with spectral grade methanol. The rinsing procedure was then repeated with toluene. Following the rinsing procedure, the beads were dried for 2 h at 180° C. then placed in an 80% humidity chamber overnight. In a glovebox under N₂ atmosphere a 1% (v/v) solution of neat DETA diluted in anhydrous toluene was prepared in an OTS silanized glass vial. In an OTS silanized 20 mL scintillation vial silica beads were then immersed in this solution, to a final volume equal to that which was originally aliquoted from the activated bead stock solution, capped and incubated at room temperature on a bench top oscillator overnight. The freshly silanized beads were then rinsed (3×) with anhydrous toluene, sonicated for 5 min and rinsed again. The rinsing procedure was repeated with spectral grade methanol and finally PBS. Beads were then taken up in PBS at a concentration of approximately 1×10⁸ beads/mL.

Recombinant human ectodomain αIIbβ3 was freshly immobilized onto silanized beads as experimentally required. Bead immobilization buffer was prepared (4 mM EDC, 10 mM sulfo-NHS, 10 mM sodium phosphate, 140 mM NaCl, 5 mM KCl pH=7.4) and recombinant human extracellular αIIbβ3 was added to a final concentration of 125 μg/mL. Immediately after the preparation immobilization buffer, freshly silanized beads were taken up in immobilization buffer to a final bead concentration of approximately 1×10⁸ beads/mL, and incubated at 4° C. overnight on a bench top oscillator. The reaction was quenched by adjusting the pH to 8.5 and incubating at RT for 1 h. The beads were then rinsed (3×) with copious amounts of PBS, sonicated for 5 minutes then rinsed one last time with PBS, the concentration was then adjusted to approximately 1×10⁸ beads/mL and then stored at 4° C. in a screw top vial until needed.

Activation of αIIbβ3 SAMs. Activation of SAM immobilized integrin αIIbβ3 was achieved by 72 h incubation in activation buffer (1 mM each of CaCl₂, MnCl₂ and MgCl₂ taken up in PBS).

X-ray Photoelectron Spectroscopy. Angle-resolved X-ray photoelectron spectroscopy (XPS) to evaluate substrate silanization (SAM formation) and subsequent αIIbβ3 immobilization was performed with a Theta probe XPS Instrument (ThermoFisher Scientific) located at Surface Interface Ontario (University of Toronto, Toronto, Ontario, Canada). Quartz surfaces were analyzed with monochromated Al Kα X-rays at takeoff angles of 27.5, 42.5, 57.5, and 72.5° relative to the normal. The binding energy scale was calibrated to the C1s signal at 285 eV. Peak fitting and data analysis were performed with the Avantage Data System software package (ThermoFisher Scientific™) provided with the instrument. Complete XPS data are tabulated Table 1.

TABLE 1
X-ray photoelectron spectroscopy (XPS) analysis
of bare, DETA silanized and αIIbβ3 immobilized
magnetic silica beads. (n ≥ 3)

| | Carbon | Nitrogen | Oxygen | Silicon |
|---|---|---|---|---|
| Surface | 293 - 280 eV | 405 - 395 eV | 543 - 527 eV | 103 - 98 eV |
| Bare | 11.08 ± 1.83^{a} | 4.79 ± 0.77^{a} | 57.49 ± 7.09 | 26.63 ± 3.02 |
| DETA | 37.02 ± 2.94 | 12.60 ± 3.29 | 35.91 ± 2.91 | 14.47 ± 1.82 |
| αIIbβ3 | 40.79 ± 2.65 | 11.04 ± 2.81 | 38.03 ± 6.26 | 10.14 ± 0.94 |

^{a}Unavoidable adventitious carbon and nitrogen contamination

Preparation of αIIbβ3 Coated Beads. Ferromagnetic silica beads were prepared upon formation of DETA adlayers on cleaned silica beads followed by covalent immobilization of aIIbβ3 (FIG. 1C). Each step of surface preparation was characterized using X-ray photoelectron spectroscopy (XPS) by following the evolution of the characteristic elements of silica (Si and O), DETA and αIIbβ3 (C and N), (see Table 1). Beside the small signal attributed to unavoidable adventitious carbon and nitrogen contamination, bare silica showed signals for oxygen and silicon at an approximate 2:1 ratio, as would be expected for quartz (SiO₂). Following DETA silanization, signals appeared for carbon and nitrogen, in contrast, the oxygen and silicon signals (that mainly originate from the now underlying silica substrate) decreased. αIIbβ integrin was then covalently coupled to the DETA surface via carbodiimide EDC/NHS chemistry. Following this step XPS signals for carbon, nitrogen and oxygen remained essentially unchanged while the silicon signal was significantly attenuated, indicating further burying of the silica substrate by αIIbβ3.

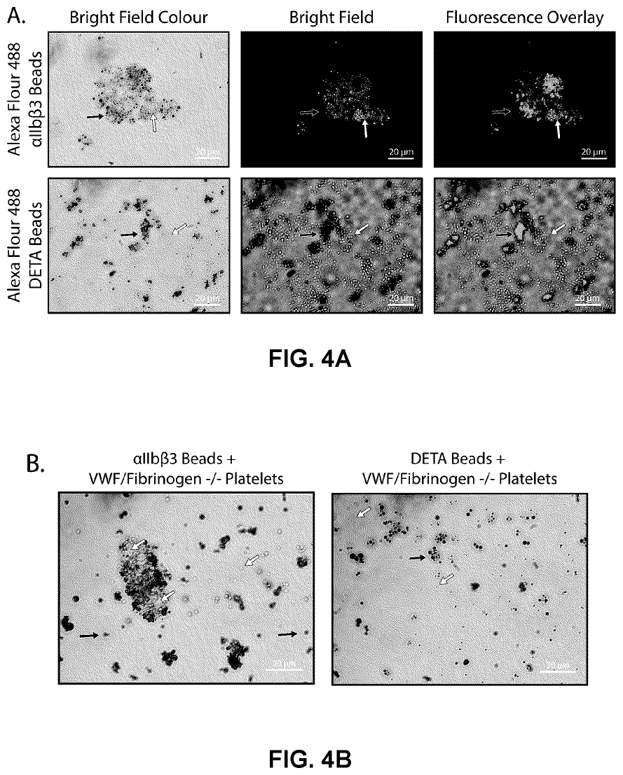

Co-aggregation of αIIbβ3 Coated Beads. Briefly, 1×10⁸ platelets/mL were incubated with 1×10⁸ αIIbβ3-coupled beads/mL and platelet aggregation was initiated with 5 U/mL of thrombin. After allowing aggregation to proceed for 45 minutes, the beads were magnetically pulled down and washed. The resulting pulled down platelet aggregates were imaged (see FIGS. 4A and B). FIGS. 4A and B clearly shows that αIIbβ3-coupled beads pulled down whole platelet aggregates while the DETA beads did not, indicating that the surface immobilized integrin is present and biologically active.

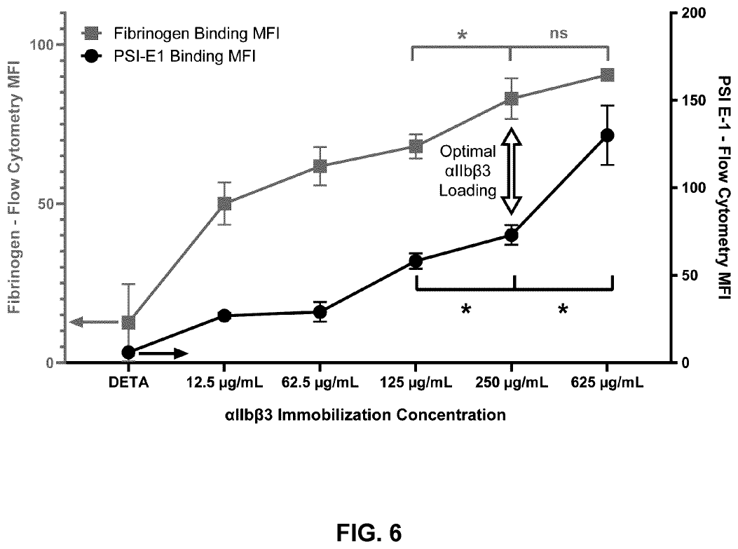

Loading optimization. At various immobilization concentrations of αIIbβ3 integrin, the resulting αIIbβ3-coupled beads were evaluated for fibrinogen or PSI-E1 binding using flow cytometry. PSI-E1 is an antibody that binds linear epitope and indicated represents the amount of integrin loaded onto the surface. While, fibrinogen is the natural ligand of αIIbβ3, and fibrinogen binding indicates the activity of the αIIbβ3-coupled beads. The optimal loading concentration was determined to be 250 μg/mL, increasing the concentration, although increases the amount of αIIbβ3 integrin on the bead surface does not increase the binding activity of the resulting αIIbβ3-coupled beads (FIG. 6).

Flow-Cytometric Analysis of Ferromagnetic Silica Beads. Unless otherwise stated, all flow cytometry experiments were conducted under the same conditions using either a BD FACS Calibur™ or BD Fortessa™ X20 flow cytometer. 3 μL of 1×10⁸ beads/mL were mixed with the desired concentration of fluorescently labelled antibody or ligand to a final volume of 100 μL in PBS and incubated for 1 h. Following incubation, the samples were diluted to a final volume of 1 mL and flow cytometrically analyzed. Samples that were compared with one another were run on the same instrument under the same instrumental conditions (signal gain, flow rate etc.). Any differences in absolute mean fluorescence intensity (MFI) value between different experiments was due to variance between the two.

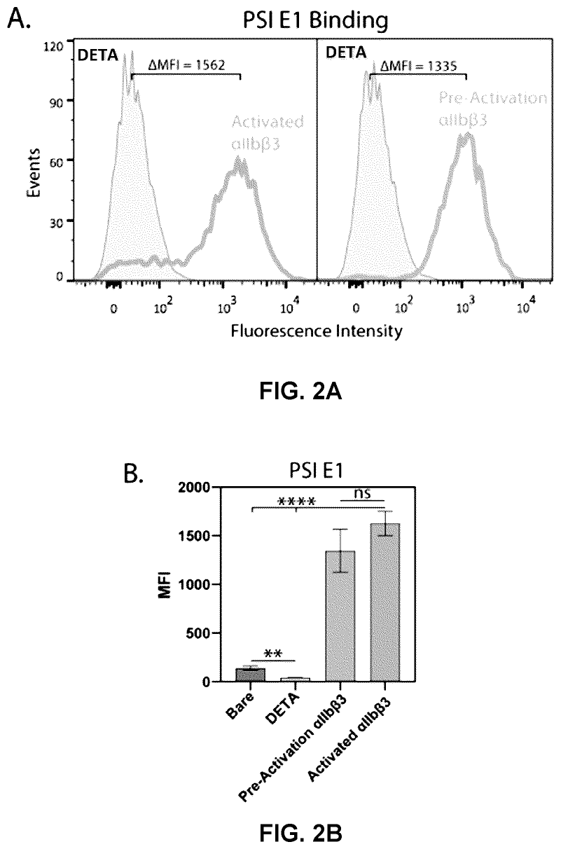

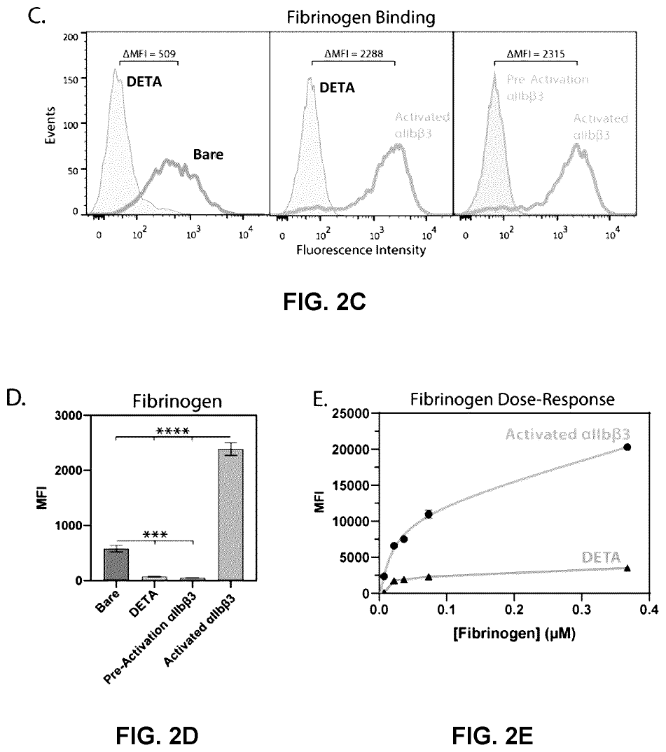

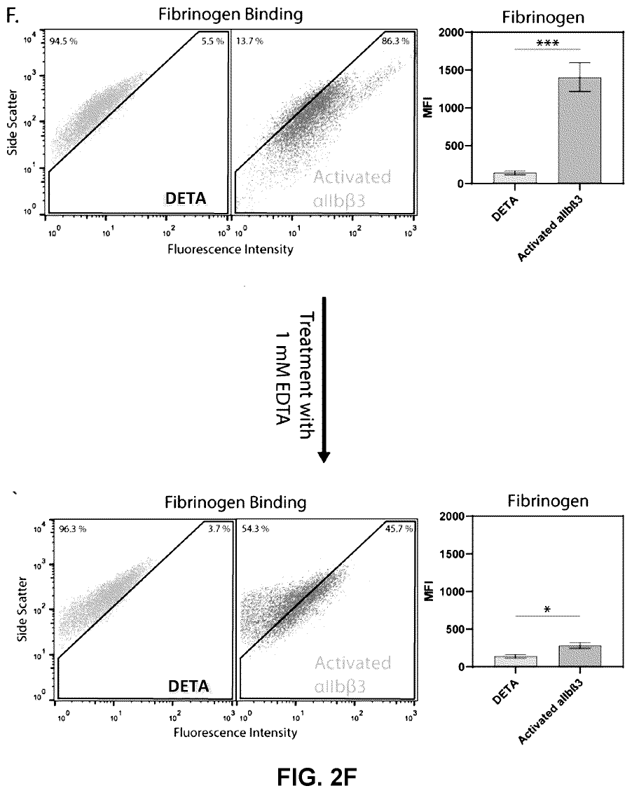

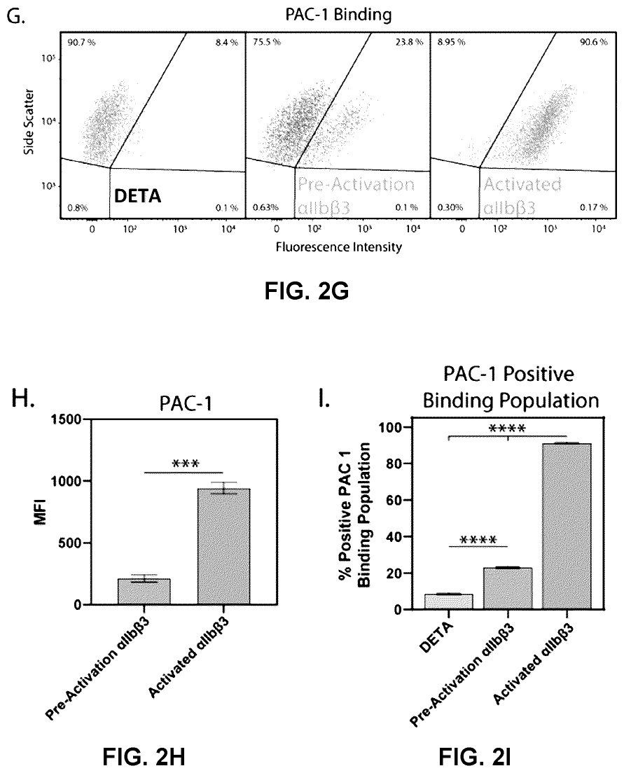

αIIbβ3 Coupled Bead Flow Cytometry Analysis. Although the XPS data suggest that the integrin deposited onto the bead surface, since αIIbβ3 lacks a characteristic element that is absent within the SAM, confirmation was necessary. Flow cytometry was utilized to analyze the beads for the presence of the integrin. αIIbβ3 coupled and DETA coated beads were incubated with FITC coupled PSI-E1, an antibody against linear epitope of the PSI-domain of β3 integrin REF GZ blood paper. Ferromagnetic silica particles at each stage of the coating fabrication process were analyzed with flow cytometry for binding to (FIGS. 2A and 2B) PSI E1, and (FIG. 2C to 2F) fibrinogen. The integrin is indeed present on the bead surface, and integrin function (ligand binding) is highly conformation dependent, and the integrin must be induced into the high affinity upright conformation prior to ligand binding. αIIbβ3 was activated by incubation with a mixture of divalent cations (Mg²⁺, Ca²⁺ and Mn²⁺) in PBS buffer. After the activation step, the beads were analyzed with flow cytometry for binding of Alexa488™ labelled fibrinogen (native αIIbβ3 ligand) and PAC-1 (an antibody specific for the active upright conformation of αIIbβ3) (See Table 2, FIGS. 2G to 2I). Fibrinogen dose-response curves were also investigated by varying the concentrations of Alexa488™ labelled fibrinogen for binding to activated αIIbβ3 and DETA (control) particles (FIG. 2E). The DETA signal was subtracted from the activated αIIbβ3 signal. The activated αIIbβ3 dose response curve was fitted to a one site specific binding model producing a K_{d} of 78±7 nM. All analyses conducted as n≥3, data presented as mean±SD, analyzed with a two-tailed t-test, ns=not significant *P<0.05, **P<0.01, ***P<0.001 and ****P<0.0001.

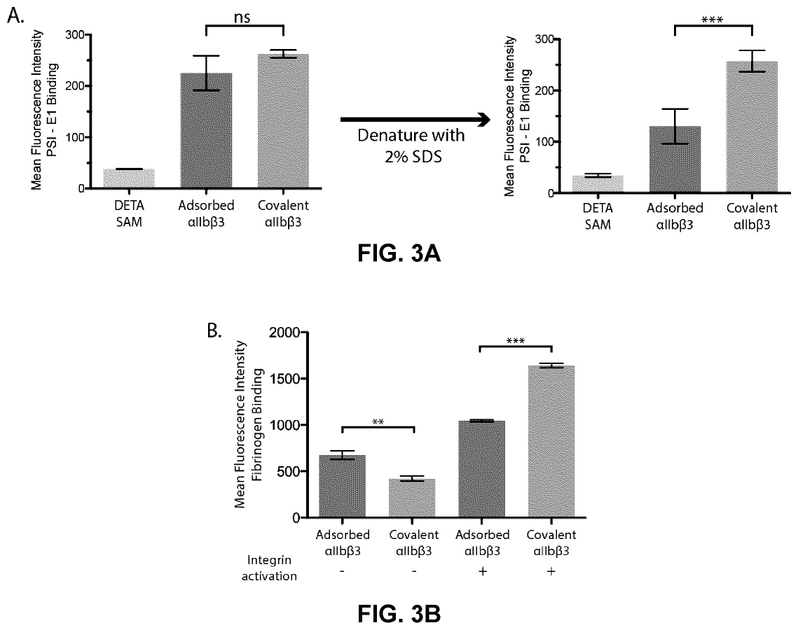

The data revealed, that indeed the covalently coupled integrin adopted the high-affinity ligand binding conformation, furthermore, the DETA coating was able to markedly resist the non-specific binding of sticky fibrinogen, a protein known to be highly fouling and clog up blood oxygenators and dialysis equipment REF, without a blocking step (i.e. BSA) when compared to the uncoated silica beads (FIG. 3C).

TABLE 2
Flow cytometry determined mean fluorescent
intensities (MFI) of bare, DETA and
activated/inactive αllbβ3 beads
bound (or nor) with alexa488-fibrinogen
(endogenous ligand), FITC-PSI E1 (conformation
independent anti-αllbβ3 antibody), FITC-PAC 1
(activated conformation specific anti-allbβ3
antibody), FITC-antiCD62 and FITC-anti GPIbβ
(Isotype controls of the various antibodies). n ≥ 3

| | |
|---|---|
| | Mean |
| | Fluorescence |
| Beads | Intensity (MFI) |

50 nM Alexa 488 - Fibrinogen

| | |
|---|---|
| Bare | 577 ± 18 |
| DETA | 74.6 ± 5.2 |
| Inactive αllbβ3 | 52.7 ± 3.9 |
| Activated αllbβ3 | 2383 ± 18 |

50 nM FITC - PAC 1

| | |
|---|---|
| Inactive αllbβ3 | 212 ± 29 |
| Activated αllbβ3 | 942 ± 28 |

50 nM FITC - PSI E1

| | |
|---|---|
| Bare | 139 ± 12 |
| DETA | 41.7 ± 3.1 |
| Inactive αllbβ3 | 1343 ± 21 |
| Activated αllbβ3 | 1625 ± 19 |

100 nM FITC - anti CD62p

| | |
|---|---|
| DETA | 52.7 ± 3.7 |
| Inactive αllbβ3 | 54.9 ± 6.0 |
| Activated αllbβ3 | 56.5 ± 4.9 |

100 nM FITC - anti GPIbβ

| | |
|---|---|
| DETA | 36.0 ± 5.1 |
| Inactive αllbβ3 | 36.7 ± 6.9 |
| Activated αllbβ3 | 40.6 ± 6.5 |

However, this data did not indicate if αIIbβ3 was covalently bound to the DETA coated bead. To evaluate this, beads were prepared in the same manner only lacking the carbodiimide crosslinker (EDC/NHS, FIG. 10 step 2) and αIIbβ3 was allowed to electrostatically and non-specifically adsorb onto the bead surface. PSI-E1 flow cytometry analysis (FIG. 3A) revealed that initially both the covalently bound and adsorbed beads loaded approximately the same amount of αIIbβ3. However, the addition of 2% SDS reduced the adsorbed bead signal by approximately 50% while the covalent bead signal remained the same, indicating that αIIbβ3 was indeed covalently bound to DETA. The adsorbed and covalently bound αIIbβ3 beads were activated and analyzed for fibrinogen (Fg) binding, and despite similar loading of αIIbβ3, the covalently bound bead produced a markedly higher signal (FIG. 3B). These data indicated that covalent attachment of αIIbβ3 enhances the active binding conformation of the integrin.

### Example II—SAM Immobilized Integrin AIIBB3 as Molecular Probes

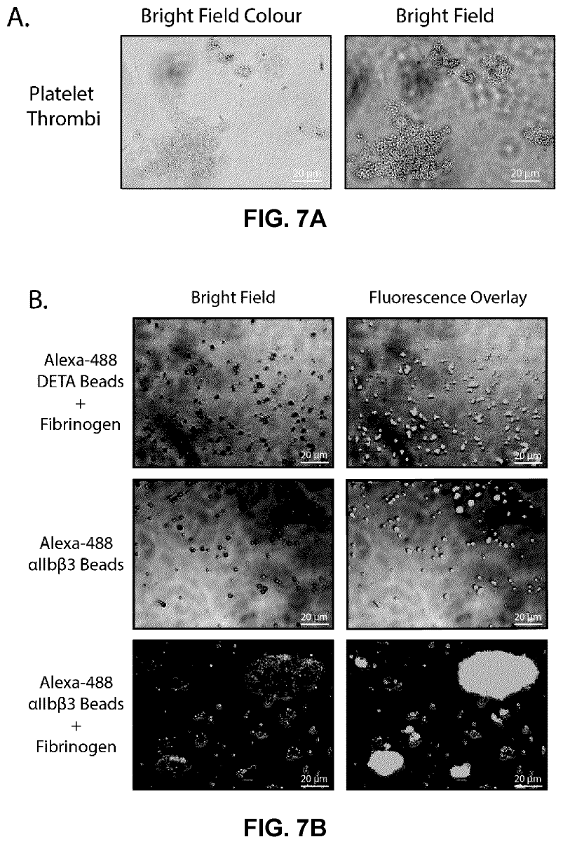

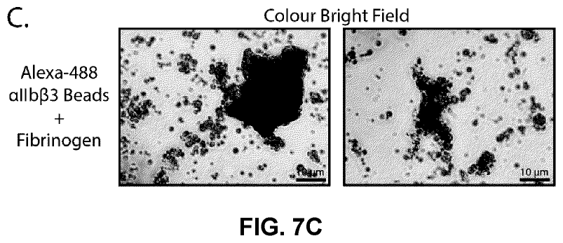

Bioactivity of αIIbβ3 Coupled Beads. DETA and αIIbβ3 coupled beads were labelled fluorescently with Alexfluor 488-NHS™ (FIG. 7A to 7C). Upon addition of fibrinogen and imaging under a fluorescence microscope (FIG. 7B), the αIIbβ3 beads were found to form aggregates while the DETA beads remained un-aggregated, indicating binding with bivalent fibrinogen.

Platelet Bead Co-aggregation Assay. DETA or activated aIIbβ3 beads were co aggregated with wild type (WT) or Fibrinogen/VWF double deficient (Fg/VWF−/−) platelets by incubating 1×10⁸/mL beads with 1×10⁸/mL gel filtered platelets in PIPES buffer (25 mM 1,4-Piperazinediethanesulfonic acid (PIPES), 140 mM NaCl, 4 mM KCl, 5.5 mM D-glucose pH 7.0). The platelets were then activated by the addition of thrombin to a final amount of 5 units/mL, followed by incubation at RT for 45 min on a rotisserie shaker. The beads were then pulled down, washed and imaged under a Zeiss Axiovert 200 inverted fluorescence microscope at 40× magnification.

Alexa-488 labelled DETA and aIIbβ3 beads were then mixed in a 1:1 ratio with gel filtered wild type (WT) murine platelets and activation was initiated with thrombin, the beads were magnetically pulled down and imaged on a fluorescent microscope (FIG. 4A). WT platelet aggregates formed under the same conditions in the absence of silica beads are depicted in FIG. 4B. The resultant microscopy images clearly show that the aIIbβ3-coupled beads were incorporated into the platelet aggregates while the DETA beads did not interact with the murine platelets. Furthermore, both unlabelled DETA and aIIbβ3 beads were co-aggregated with vWF/fibrinogen −/− platelets under the same conditions as previous, and FIG. 4B clearly demonstrates that the incorporation of aIIbβ3 coupled beads into the double knock out platelets, indicating that the beads do interact with the yet unidentified ligands that mediate fibrinogen-independent platelet aggregation.

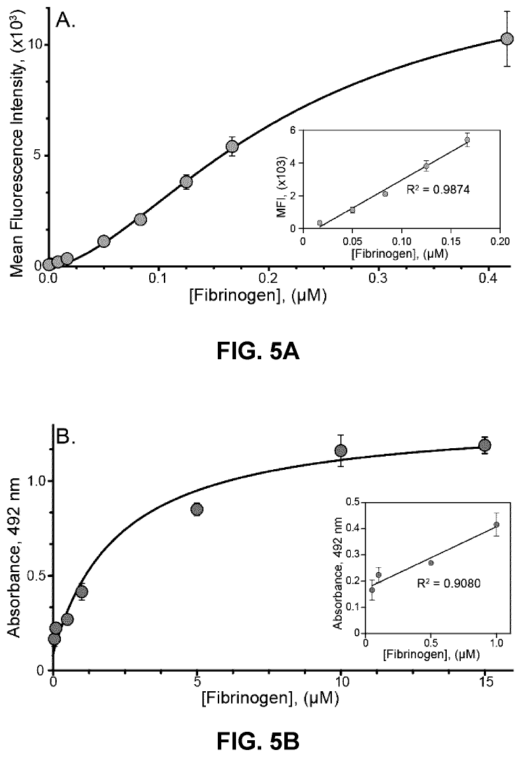

Fibrinogen ELISA Assay. To ensure similar immobilization levels of recombinant human ectodomain aIIbβ3 between the ELISA plate and integrin coupled magnetic beads, the wells of a 96-well micro plate (Nunc MaxiSorp) were incubated with the same aIIbβ3 concentration per surface area as during the preparation of the magnetic beads, 6.6×10⁴ μg·mL⁻¹·c⁻². Each well was coated with aIIbβ3 or control proteins (BSA and β3−/− platelet lysate) by incubation of 6 μg/μL protein in binding buffer (TRIS buffered saline with 0.05% TWEEN-20 and 1 mM each of MgCl₂, MnCl₂ and CaCl₂)) at 4° C. overnight. Incubation of 3% skim milk (ED Millipore) and 2% TWEEN-20 for 1 hour at 37° C. was used for blocking. See FIGS. 5A and 5B. The concentration of aIIbβ3 per surface area of bead used when coating SAM coated beads is 6.6×10⁴ μg·mL⁻¹·cm⁻², therefore the wells of 96 well plates used were coated with the same density EDC/NHS quenching achieved by increase pH to 8.6. Bead concentration for synthesis: 6×10⁹ beads·mL⁻¹; Integrin-bead reaction concentration=250 μg·mL⁻¹; Integrin Used=6.6×10⁴ μg·mL⁻¹·cm⁻²; Well Surface Area (50 μL)=0.93 cm²; Concentration/wee for ELISA=6×10⁴ μg·mL⁻¹ (6 μL/well in 50 μL total volume); Stock [αIIbβ3]=5×10⁵ μg·mL⁻¹.

Fibrinogen binding. Wells were incubated with the desired fibrinogen concentration in binding buffer for 1 hour at 37° C. Wells were then incubated with anti-fibrinogen mouse IgG (Sigma Aldrich) followed by incubation with anti-mouse IgG—horseradish peroxidase (HRP) (Santa Cruz), both at 37° C. for 1 h. Peroxidase substrate, o-Phenylenediamine dihydrochloride (OPD, Sigma Aldrich) was prepared at 0.4 mg/mL in 0.05 M phosphate citrate buffer at pH=5.0 with 0.4 μL/mL of 30% H₂O₂. The OPD peroxidase reaction was stopped after 1 hour with 2 M H₂SO₄ and the absorbance was read at 492 nm. Between incubation steps the plate was washed with copious amounts of TRIS buffered saline with 0.5% TWEEN-20.

Fibrinogen Detection, αIIbβ3 Coupled Beads vs ELISA. To evaluate the analytical potential of these αIIbβ3 coupled molecular probes, a flow cytometry sandwich assay was developed. In literature reports the most common method utilized to detect integrin binding interactions is ELISA, hence, the performance of the flow cytometry-based assay (FIG. 5A) was tested against traditional sandwich ELISA (FIG. 5B). ELISA was performed by immobilizing the same density of integrin (amount of integrin per surface area, bead or plate) onto the plate surface followed by blocking with 5% skim milk with 0.5% TWEEN, while the flow cytometry assay did not include a blocking step. The same primary antibody was used for both assays, however, the detection antibody for ELISA was HRP labelled and for flow cytometry was FITC labelled. The fibrinogen does response curves are depicted in the inserts of each of FIGS. 5A and 5B. The flow cytometry-based assay produced a Kdₐₚₐᵣₐₙₜ of 0.21±0.03 μM and CLOD of 0.026±0.002 μM while ELISA produced a K_{d} of 2.2±0.4 μM and CLOD of 0.54±0.07 μM. The flow cytometry based assay produced a significant increase in performance compared to ELISA, even though a blocking step and a signal amplification-based detection strategy were employed in ELISA. It was postulated that the significant increase in performance observed is due to the SAM and the immobilization strategy which promotes the high-affinity ligand binding conformation of the integrin.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

1. A surface having a first and a second hydroxyl group and at least one self-assembled multimer immobilized thereon, wherein:
the at least one self-assembled multimer comprises at least one of a first chimeric polypeptide associated with a second chimeric polypeptide,
the first chimeric polypeptide is of formula (Ia) or (Ib):
NH₂-FPM-FAAL-AAT-COOH (Ia)
NH₂-AAT-FAAL-FPM-COOH (Ib)
wherein FPM is a first polypeptide moiety;
FAAL is an optional first amino acid linker;
AAT is an acidic amino acid tail having at least three acidic amino acid residues each having an R-group comprising a carboxyl group, and wherein the AAT has a pI between about 3 and about 5; and
— is an amine bond;
the second chimeric polypeptide is of formula (IIa) or (IIb):
NH₂-SPM-SAAL-BAT-COOH (IIa)
NH₂-BAT-SAAL-SPM-COOH (IIb)
wherein SPM is a second polypeptide moiety;
SAAL is an optional second amino acid linker;
BAT is a basic amino acid tail having at least one acid amino acid residue having an R-group comprising a carboxyl group, and wherein the BAT has a pI between about 9 and about 11; and
— is an amine bond;
the carboxyl group of the first chimeric polypeptide is covalently associated to a first silane linker (FSL) moiety, wherein the FSL is covalently associated with a first hydroxyl group of the surface;
the carboxyl group of the second chimeric polypeptide is covalently associated to a second silane linker (SSL) moiety, wherein the SSL is covalently associated with a second hydroxyl group of the surface;
the AAT is non-covalently associated with the BAT; and
the first chimeric polypeptide is non-covalently associated with the second polypeptide moiety.

2. The surface of claim 1, wherein the AAT is at least three and up to 50 amino acid residues in length.

3. The surface of claim 1, wherein the AAT has a pI is about 4.

4. The surface of claim 3, wherein the AAT has a pI of about 3.91.

5. The surface of claim 4, wherein the AAT has an amino acid sequence of SEQ ID NO: 4 or functional variants or fragments thereof.

6. The surface of claim 1, wherein the BAT is at least one and up to 50 amino acid residues in length.

7. (canceled)

8. The surface of claim 1, wherein the BAT has a pI of about 10.1.

9. The surface of claim 8, wherein the BAT has an amino acid sequence of SEQ ID NO: 9 or functional variants or fragments thereof.

10. The surface of claim 1,
wherein the first chimeric polypeptide has the FAAL; and/or
wherein the second chimeric polypeptide has the SAAL.

11. The surface of claim 1, wherein the at least one self-assembled multimer comprising an ectodomain of a surface protein.

12. The surface of claim 11, wherein one or more of the at least one self-assembled multimer is an activated surface protein.

13. The surface of claim 11, wherein the at least one self-assembled multimer is an integrin dimer.

14. The surface of claim 13, wherein the first chimeric polypeptide comprises a αIIb polypeptide, and the second chimeric polypeptide comprises a β3 polypeptide.

15. The surface of claim 14, wherein the FPM has an amino acid sequence of SEQ ID NO: 2 or functional variants or fragments thereof.

16. The surface of claim 14, wherein the SPM has an amino acid sequence of SEQ ID NO: 7 or functional variants or fragments thereof.

17. The surface of claim 1, wherein the surface is a spherical surface or a planar surface.

18. The surface of claim 17, wherein the surface is a microsphere silica bead.

19. (canceled)

20. (canceled)

21. The surface of claim 1, wherein the FSL and/or SSL comprise one or more amine or thiol groups that are covalently associated with the carboxyl groups of the AAT or the BAT.

22. The surface of claim 21, wherein the FSL and/or SSL moieties comprise (3-trimethoxysilylpropyl) diethylenetriamine (DETA).

23. The surface of claim 1, comprising:
the first chimeric polypeptide of formula (Ia) and the second chimeric polypeptide of formula (IIa);
the first chimeric polypeptide of formula (Ib) and the second chimeric polypeptide of formula (IIa);
the first chimeric polypeptide of formula (Ia) and the second chimeric polypeptide of formula (IIb); or
the first chimeric polypeptide of formula (Ib) and the second chimeric polypeptide of formula (IIb).

24. A process of immobilizing at least one self-assembled multimer to a surface having a first and a second hydroxyl group covalently associated with a first and a second silane linker moiety, the at least one self-assembled multimer comprising at least one of a first and a second chimeric polypeptide, the process comprising:
obtaining the first chimeric polypeptide as defined in claim 1;
obtaining the second chimeric polypeptide as defined in claim 1; and
adding the first and the second chimeric polypeptide to the surface in a solvent under suitable conditions for the first and the second chimeric polypeptides to covalently bond to the surface via the silane linker moieties;
wherein the AAT of the first chimeric polypeptide is non-covalently associated with the BAT of the second chimeric polypeptide and the first polypeptide moiety is non-covalently associated with the second polypeptide moiety.

25. The process of claim 24, wherein the first and second silane linker moieties comprise one or more amine or thiol groups that are covalently associated with the carboxyl groups of the AAT or BAT.

26. (canceled)

27. The process of claim 24, further comprising coating the surface with the silane linker moieties by reacting with the hydroxyl groups.

28. The process of claim 24, further comprising obtaining the first and the second chimeric polypeptide from recombinant expression in a recombinant host cell.

29. The process of claim 24, further comprising activating the at least one self-assembled multimer.

30. (canceled)

31. (canceled)

32. The process of claim 24, wherein the at least one self-assembled multimer comprises an ectodomain a surface protein.

33. The process of claim 32, wherein the at least one self-assembled multimer is an integrin dimer.

34. (canceled)

35. (canceled)

36. A kit comprising (i) a first and (ii) a second chimeric polypeptide as defined in claim 1, wherein the first and the second chimeric polypeptide are capable of forming a multimer and optionally (iii) a surface for covalently associating the first and the second chimeric polypeptide, wherein the surface has hydroxyl groups covalently associated with a first and a second silane linker moiety.

37. The kit of claim 36, wherein:
the first chimeric polypeptide has a first polypeptide moiety (FPM), an optional first amino acid linker (FAAL), and a first amino acid tail (AAT) having at least three acid amino acid residue having an R-group comprising a carboxyl group, and wherein the AAT has a pI between about 3 and 5; and
the second chimeric polypeptide has a second polypeptide moiety (SPM), an optional second amino acid linker (SAAL), and a second amino acid tail (BAT) having at least one acid amino acid residue having an R-group comprising a carboxyl group, and wherein the BAT has a pI between about 9 and 11.

38. The kit of claim 37, wherein the FPM is a αIIb polypeptide and the SPM is a β3 polypeptide.

39. The kit of claim 38, wherein the first chimeric polypeptide has an amino acid sequence of SEQ ID NO: 1 or functional variants or fragments thereof.

40. The kit of claim 39, wherein the second chimeric polypeptide has an amino acid sequence of SEQ ID NO: 6 or functional variants or fragments thereof.

41. (canceled)

42. (canceled)

43. (canceled)

44. (canceled)

45. (canceled)
